(19) United States
(12) Patent Application Publication
(10) Pub. No.: US 2008/0166307 A1
(43) Pub. Date: Jul. 10, 2008

(54) Oral Care Compositions Comprising a Hippophae Extract

(76) Inventors: Jose Eder Fontana, Sao Paulo (BR); Marcos Roberto Bettani, Sao Bernardo do Campo (BR); Alina Emirova, Moscow (RU); Ieda Ferreira, Sao Paulo (BR); Joao Abel Correia, Santo Andre (BR); Paulo Focassio, Sao Bernardo do Campo (BR); Eduardo Carlos Vernilho de Lima, Sao Paulo (BR)

Correspondence Address: COLGATE-PALMOLIVE COMPANY, 909 RIVER ROAD, PISCATAWAY, NJ, 08855, US

(21) Appl. No.: 11/621,846

(22) Filed: Jan. 10, 2007

(51) Int. Cl. A61K 8/97

(52) U.S. Cl. 424/58

(57) ABSTRACT

Oral care compositions that comprise a Hippophae extract, as well as methods for reducing inflammation, inhibiting gingivitis, improving oral hygiene and maintaining or increasing systemic health of a mammal, are disclosed.

### BACKGROUND OF THE INVENTION

Gum disease is associated with the accumulation of bacteria in the oral cavity. Gingivitis is an early phase of gum disease and is characterized by an inflammation of the gums caused by the accumulation of plaque, a soft, sticky, colorless film of bacteria above the gum line. If not routinely removed by proper brushing and flossing, plaque tends to build up on teeth and gums, exacerbating gingivitis over time and leading to more serious gum diseases such as periodontitis. Periodontitis is believed to occur where unremoved plaque hardens into calculus (tartar), affecting periodontal ligaments and eventually leading to destruction of bone and loss of teeth.

Nearly 80% of American adults have some form of gum disease. Gum disease can be generally treated and minimized with a fairly straightforward dental hygiene regimen, including regular brushing, flossing and professional dental cleanings. However, this hygiene routine is not always strictly followed. Thus, there is a relatively high presence of gum disease in the adult population.

Thus, it is desirable to enhance the anti-gingivitis activity of oral care compositions such as dentifrices, such that the routine brushing of teeth may have maximum impact of treating and eliminating the occurrence of gum disease. There is an ongoing need for oral care compositions that effectively combat plaque and diseases of the oral cavity. Additionally, oral compositions that contain natural ingredients are desirable.

### BRIEF SUMMARY OF THE INVENTION

In certain embodiments, the present invention provides oral compositions comprising about 0.001 to about 20% of a Hippophae extract and an acceptable carrier.

In other embodiments, the present invention provides methods of reducing inflammation and inhibiting gingivitis, comprising applying a composition comprising about 0.001 to about 20% of a Hippophae extract and an acceptable carrier to the oral surfaces.

In other embodiments, the present invention provides methods of improving oral hygiene comprising the application of a composition comprising about 0.001 to about 20% of a Hippophae extract and an acceptable carrier to the oral surfaces.

In other embodiments, the present invention provides methods of maintaining or increasing the systemic health of a mammal comprising applying a composition to an oral surface of a mammal at least once a day for a duration of time, wherein the composition comprises about 0.001 to about 20% of a Hippophae extract and an acceptable carrier.

### DETAILED DESCRIPTION OF THE INVENTION

As used throughout the present disclosure, ranges are a shorthand for describing each and every value that is within the range. Any value within the range can be selected as the terminus of the range. In addition, all references cited in the present disclosure are hereby incorporated by reference in their entireties. Where there is a conflict between definitions provided in such a reference with those of the present invention, the definition in the present invention controls herein.

The present invention relates, in certain embodiments, to oral care compositions that provide anti-inflammatory and anti-gingivitis benefits. The present invention is particularly useful with respect to dentifrice compositions.

In certain embodiments, the Hippophae extract is provided together with conventional components found in oral care compositions. The Hippophae extract component and other components will be described herein.

Plants of the genus Hippophae include Hippophae rhaminoides (“common sea buckthorn” or “sea berry”), Hippophae salicifolia (“willow-leaved sea buckthorn”), Hippophae tibetana (“Tibetan sea buckthorn”), as well as other varieties such as, for example, Hippophae goniocarpa, Hippophae neurocarpa, Hippophae turkistanica, Hippophae caucasica and Hippophae mongolica.

A Hippophae extract may be derived from any part of the plant, including the berries, leaves, roots, stems and seeds. Primarily, a Hippophae extract has been described as having anti-inflammatory properties, and is reportedly an excellent source of vitamin A and other carotenes, vitamin E and other tocopherols, flavonoids, vitamin C, vitamins B1 and B2 and vitamin K. Hippophae extract has also been shown to be rich in essential fatty acids, plant sterols, lignans and minerals.

As used herein, “extracting or “extraction” of a solid or liquid material refers to contacting the material with an appropriate solvent to remove the substance(s) desired to be extracted from the material. Where the material is solid, it may be cleaned of debris and excess wax, and then broken into smaller pieces and crushed or ground to a powder prior to contacting it with a solvent. Extraction may be carried out by conventional means known to a skilled artisan, e.g., by using an extraction apparatus such as a Soxhlet apparatus, which retains the solid material in a holder and allows the solvent to flow through the material; by blending the solvent and material together and then separating the liquid and solid phases or two or more immiscible liquid phases, such as by filtration or by settling and decanting. Preparation of the extract can be carried out by other conventional means, such as, e.g., supercritical extraction (e.g., supercritical fluid extraction using CO₂), steam distillation or using vehicles such as sunflower or avocado oils.

As used herein, “extract” refers to the resultant solid or liquid material from an extraction. It is preferred that the natural extract active ingredients used in the oral care compositions of the present invention are of reproducible, stable quality and have microbiological safety. The natural extracts useful for the present invention, including the Hippophae extract, may be made from any part of the plant, including the leaves, fruit, pulp, seeds, stems, roots or any mixture thereof.

In certain embodiments, the compositions of the present invention comprise about 0.001 to about 20% of a Hippophae extract. In other embodiments, the compositions comprise about 0.002 to about 15%, about 0.005 to about 12%, about 0.01 to about 10% or about 0.015 to about 5% of a Hippophae extract.

Analytical determination using GC-MS for fatty acid distribution and HPLC for vitamin E has been done in samples obtained by different extraction processes and parts of the plants. Fatty acid distribution is characteristic for pulp as well as for seed oils, according to the range below, shown in Table 1:

| Component | Weight Percentage Range |
|---|---|
| Palmitic acid (C16:0) | 8.0 to 40.0 |
| Palmitoleic acid (C16:1) | 0.5 to 50.0 |
| Stearic acid (C18:0) | 0.5 to 40.0 |
| Oleic acid (C18:2) | 3.0 to 60.0 |
| Linoleic acid (C18:2) | 0.01 to 40.0 |
| Linolenic acid (C18:3) | 0.01 to 25.0 |

Vitamin E levels ranged from about 800 mg/100 g to about 2,600 mg/100 g, where pulp fruits oils presented more content.

Preferred embodiments of the present invention are directed to compositions comprising a Hippophae extract in an acceptable carrier. “Acceptable carrier” as used throughout, is interchangeable with “acceptable vehicle” and refers to a carrier with which the Hippophae extract may be combined, commensurate with a reasonable benefit/risk ratio, to achieve a specific and desired dosage form. Generally, the acceptable carrier should not substantially degrade or otherwise adversely affect the extract. Additionally, the acceptable carrier should be one that does not adversely affect the oral surfaces to which it may be applied, and is otherwise acceptable for administration to the oral surfaces of a patient. Conventional carriers and vehicles used in oral care compositions such as dentifrices are known to a skilled artisan and may be used in the present invention.

As used herein, “oral surfaces” refers to the oral mucosa of a patient, and includes the teeth, gingiva, palate, tongue and other tissues within the oral cavity.

In some embodiments, the present invention is directed to a dentifrice composition. Dentifrice compositions of the present invention may include, but are not limited to: toothpastes, tooth gels, tooth polishes, tooth powders, mouthwashes and mouthrinses. In certain embodiments, the dentifrice is a toothpaste. In other embodiments, the composition is capable of being impregnated in, or deposited on, a dental floss or dental tape. In such embodiments, the compositions may further comprise a wax, such as, e.g., a microcrystalline wax. In certain embodiments, such wax is present in amounts of about 1% to about 18%, in other embodiments about 5% to about 15%, in other embodiments about 8% to about 13% of the composition.

Materials useful in acceptable carriers include but are not limited to: adhesion agents, viscosity modifiers, diluents, surfactants, foam modulators, peroxide activators, peroxide stability agents, abrasives, pH modifying agents, humectants, mouth feel agents, sweeteners, flavorants, colorants, and combinations thereof. Carriers that may be used to prepare the oral care compositions of the present invention will, in certain embodiments, comprise a water phase containing a humectant therein. The humectant may be, for example, glycerin, sorbitol, xylitol, and/or propylene glycol of molecular weight in the range of 200-1000, but other humectants and mixtures thereof may also be employed. Reference herein to sorbitol refers to the material typically commercially available as a 70% aqueous solution.

Water is present typically in amounts of at least about 10% by weight, and generally about 25% to about 70% by weight of a dentifrice composition of the present invention. Where the dentifrice is a toothpaste, the water employed in its preparation may be deionized and free of organic impurities. The amounts of water defined herein include the free water which is added plus that which is introduced with other materials, such as with the sorbitol. In one embodiment, the dentifrice composition has a pH of at least about seven.

The dentifrice compositions of the present invention may contain a variety of optional dentifrice ingredients. As described below, such optional ingredients may include, but are not limited to: a thickening agent, a calcium ion donor, a surfactant, a fluoride ion source, a synthetic anionic polycarboxylate, a flavoring agent, an abrasive, an additional anti-plaque agent and a coloring agent.

Thickening agents or thickeners that may be used in the compositions of the present invention include natural and synthetic gums and colloids, examples of which include carrageenan (rich moss), xanthan gum and sodium carboxymethyl cellulose, starch, polyvinyl pyrrolidone, hydroxyethyl propyl cellulose, hydroxybutyl methyl cellulose, hydroxypropyl methyl cellulose, and hydroxylethyl cellulose. Inorganic thickeners include amorphous silica compounds that function as thickening agents, e.g., colloidal silica compounds available under trademarks including Cab-o-Sil, fumed silica manufactured by Cabot Corporation and distributed by Lenape Chemical, Bound Brook, N.J., USA; Zeodent 165 from J. M. Huber Chemicals Division, Havre deGrace, Md., USA; and Sylox 15, also known as Sylodent 15, available from Davidson Chemical Division of W.R. Grace Corporation, Baltimore, Md., USA. In certain embodiments, the thickening agent is generally present in the dentifrice composition in an amount of about 0.1% to about 10% by weight; in other embodiments about 0.5% to about 4% by weight of the composition.

In certain embodiments, the oral care compositions of the present invention further comprise a calcium ion donor. As used herein, the term “calcium ion donor” refers to any compound that is capable of providing calcium ions. Examples of calcium ion donors that are suitable for the present invention include calcium salts, such as, e.g., calcium glycerophosphate, calcium phosphate, calcium gluconate, calcium carbonate, calcium chloride, calcium sulfate, calcium oxide or mixtures thereof. In certain embodiments, the calcium ion donor is calcium glycerophosphate. In certain embodiments, the calcium ion donor is precipitated calcium carbonate.

In certain embodiments, the calcium ion donor is present in an amount of about 0.001 to about 60%, in other embodiments about 0.05 to about 58%, and in other embodiments about 0.08 to about 55%.

Surfactants may be used in the compositions of the present invention to achieve increased prophylactic action and/or render dentifrice compositions more cosmetically acceptable. Useful surfactants for the present invention are detersive materials that impart to the composition detersive and foaming properties.

Surfactants are frequently anionic, although other surfactants such as nonionic surfactants may also be used. Examples of suitable surfactants include water-soluble salts of higher fatty acid monoglyceride monosulfates, such as the sodium salt of monosulfated monoglyceride of hydrogenated coconut oil fatty acids; higher alkyl sulfates such as sodium lauryl sulfate; alkyl aryl sulfonates such as sodium dodecyl benzene sulfonate; higher alkyl sulfoacetates, such as sodium lauryl sulfoacetate; higher fatty acid esters of 1,2-dihydroxypropane sulfonate; and the substantially saturated higher aliphatic acyl amides of lower aliphatic amino carboxylic compounds, such as those having 12-16 carbons in the fatty acid, alkyl or acyl radicals; and the like. Examples of the last mentioned amides include N-lauryl sarcosine, and the sodium, potassium and ethanolamine salts of N-lauryl, N-myristoyl, or N-palmitoyl sarcosine.

A surfactant is typically present in the dentifrice compositions of the present invention in an amount of about 0.3% to about 5% by weight in some embodiments, about 0.5% to about 2% by weight in other embodiments.

Nonionic surfactants may also be utilized in the dentifrice compositions of the present invention. Those materials include, for example, nonanionic polyoxyethylene surfactants, such as Polyoxamer 407, Steareth 30, Polysorbate 20, and PEG-40 Castor Oil; and amphoteric surfactants, such as cocamidopropyl betaine (tegobaine), and cocamidopropyl betaine lauryl glucoside; condensation products of ethylene oxide with various hydrogen containing compounds that are reactive therewith and have long hydrocarbon chains (e.g., aliphatic chains of from about 12 to about 20 carbon atoms), which condensation products (ethoxamers) contain hydrophilic polyoxyethylene moieties, such as condensation products of poly(ethylene oxide) with fatty acids, fatty alcohols, fatty amides and other fatty moieties, and with propylene oxide and polypropylene oxides (e.g., Pluronic™ materials).

Where the compositions of the present invention are dentifrice compositions, they may also contain a source of fluoride ions or a fluorine-providing component as an anticaries agent, in an amount sufficient to supply about 25 ppm to about 5,000 ppm of fluoride ions. Preferred fluoride ion sources include inorganic fluoride salts, such as soluble alkaline metal salts; for example, sodium fluoride, potassium fluoride, sodium fluorosilicate, ammonium fluorosilicate, sodium monofluorophosphate; as well as tin fluorides such as stannous fluoride.

In addition to fluoride compounds, the oral care compositions of the present invention may also include anticalculus agents such as pyrophosphate salts. These may include dialkali or tetraalkali metal pyrophosphate salts such as Na₄P₂O₇, K₄P₂O₇, Na₂K₂P₂O₇ Na₂H₂P₂O₇, and K₂H₂P₂O₇. Other anticalculus agents that may be used include long-chain polyphosphates such as sodium hexametaphosphate, and cyclic phosphates such as sodium trimetaphosphate. In certain embodiments, these agents are included in the dentifrice compositions in concentrations of about 1% to about 5% by weight.

Antibacterial agents are additional active agents that may be useful in the compositions of the present invention. If present, an antibacterial agent is present in certain embodiments in amounts of about 0.2% to about 1% by weight of the composition. Such useful antibacterial agents include non-cationic antibacterial agents that are based on phenolic or bisphenolic compounds, such as halogenated diphenyl ethers, e.g., triclosan (2,4,4′-trichloro-2′-hydroxydiphenyl ether).

Synthetic anionic polycarboxylates may also be used in the dentifrice compositions of the present invention as an efficacy enhancing agent for any antibacterial, anticalculus or any other active agent within the dentifrice compositions. Such anionic polycarboxylates are generally employed in the form of their free acids or, in certain embodiments, partially or more preferably fully neutralized water soluble or water swellable (hydratable, gel forming) alkali metal (for example, potassium and sodium) or ammonium salts. Certain embodiments of the present invention include 1:4:1 copolymers of maleic anhydride or acid with another polymerizable ethylenically unsaturated monomer, preferably methyl vinyl ester maleic anhydride having a molecular weight (MW) of about 30,000 to 1,800,000, more preferably about 30,000 to 700,000. Examples of these copolymers are available from GAF Corporation under the trade name Gantrez; for example, AN139 (MW=500,000), AN119 (MW=250,000); S-97 pharmaceutical grade (MW=700,000), AN169 (MW=1,200,000-1,800,000), and AN179 (MW=above 1,800,000). In certain embodiments, the polymer is S-97 pharmaceutical grade (MW=700,000). Further examples of polymers that are useful for the embodiments of the present invention include polymers such as those described in U.S. Pat. No. 5,344,641 to Gaffar et al. When present, the anionic polycarboxylate should be employed in amounts effective to achieve the desired enhancement of the efficacy of any antibacterial, anti-calculus or other active agent within the dentifrice composition. Generally, the anionic polycarboxylate is present within the dentifrice composition in amounts of about 0.05% to about 4% by weight; more preferably, about 0.5% to about 2.5% by weight of the composition.

The oral care compositions of the present invention may include abrasives, such as, e.g., precipitated silicas having a mean particle size of up to about 20 microns. Examples of such compounds include Zeodent 115, marketed by J.M. Hubert Chemical Division (Havre deGrace, Md., USA) and Silodent, 783, marketed by Davison Chemical Division of W.R. Grace and Company (USA).

Other useful dentifrice abrasives include, without limitation: metaphosphate, potassium metaphosphate, tricalcium phosphate, dihydrated dicalcium phosphate, aluminum silicate, calcined alumina, bentonite, and other silaceous materials, or combinations thereof. Specific embodiments of abrasive materials useful in the present invention include, e.g., silica gels and precipitated amorphous silicas in certain embodiments having an oil absorption value of less than about 100 cc/100 g silica and, in other embodiments, in the range of about 45 cc/100 g to less than about 70 cc/100 g silica. These silicas are colloidal particles having an average particle size of about 3 microns to about 12 microns, and a pH of about 4 to about 10 when measured as a 5% by weight slurry.

Oil absorption values are measured using the ASTM Rub-Out Method D281. When present in the oral compositions of the present invention, the low oil absorption silica abrasives are present in a concentration of about 5% to about 40% by weight in certain embodiments, about 10% to about 30% by weight in other embodiments. Examples of low absorption silica abrasives that may be used in the present invention include those that are marketed under the trade designation Sylodent XWA by Davison Chemical, a division of W.R. Grace and Company (Baltimore, Md., USA); and Sylodent 650 XWA, a silica hydrogel, composed of particles of colloidal silica, having a water content of 29% by weight, averaging from about 7 to about 10 microns in diameter, and having an oil absorption of less than 70 cc/100 g of silica.

The oral care compositions of the present invention may also contain one or more flavoring agents. Flavoring agents that are useful for the present invention include, but are not limited to the following: essential oils, as well as various flavoring aldehydes, esters, alcohols, and similar materials. Examples of the essential oils include, but are not limited to, oils of spearmint, peppermint, wintergreen, sassafras, clove, sage, eucalyptus, marjoram, cinnamon, ginger, lemon, lime, grapefruit, grape, orange, apple, pear, peach, strawberry, cherry, vanilla, miswak, cola, apricot, melon, banana, coffee, cocoa, papaya, mango, kiwi, pineapple, menthol and honey. Also useful are such chemicals as menthol, carvone, and anethole.

In certain embodiments, the flavoring agent is incorporated in the dentifrice composition at a concentration in the range of about 0.05% to about 5% by weight; in other embodiments about 0.1% to about 3% by weight; in other embodiments about 0.5 to about 1.5% by weight.

Various other materials may be incorporated into the oral care compositions of the present invention. These may include, but are not limited to: desensitizers, such as potassium nitrate; whitening agents, such as hydrogen peroxide, calcium peroxide, and urea peroxide; preservatives; silicones; colorants such as pigments or dyes; and chlorophyll compounds. Sweeteners may also be included, such as, e.g., sodium saccharin, in amounts of about 0.001 to 0.5% in certain embodiments, 0.002 to 0.3% in other embodiments.

Furthermore, other active agents may be additionally included in the present compositions. Such active agents may include, but are not limited to: antioxidants, vitamins and other plant extracts. Examples include, but are not limited to: chamomile, sage, tea tree, almond, Echinacea, berries, burdock, elderberry, flax seed, ginger, ginseng, green tea, goldenseal or aloe vera). The compositions may further comprise additional polymers such as, e.g., chitosan. These additional active agents, if present, are included in the compositions in amounts of about 0.001 to about 5% in some embodiments, about 0.005 to about 3% in other embodiments, about 0.01 to about 1% in yet other embodiments.

In addition, in embodiments directed to a dental floss comprising the compositions of the present invention, wax may additionally be present. As used herein, “comprising” includes mixed with, coated on, impregnated, deposited on, adsorbed on and the like.

In certain embodiments, the present invention is directed to oral care compositions such as, e.g., mouth pastes or mouth gels and sprays that have therapeutic effect, for example, provide relief against canker sores, lesions and other mouth sores, as well as swelling and inflammation of the oral surfaces. In such embodiments, additional ingredients may include local anesthetics, such as, for example, benzocaine, lidocaine, procaine, tetracaine or tridocaine.

When present, all additional materials are incorporated into the oral care composition in their conventional amounts, and preferably in amounts that provide their desired benefits but do not substantially adversely affect the properties and characteristics desired for the oral care composition itself.

The preparation of a dentifrice is well known in the art, and is described, for example, in U.S. Pat. Nos. 3,966,863; 3,980,767; 4,328,205; and 4,358,437. More specifically, to prepare a dentifrice of the present invention, generally, the humectant (e.g., glycerin, sorbitol, propylene glycol, and/or polyethylene glycol) is dispersed in water in a conventional mixer under agitation. Into that dispersion are added the organic thickeners, such as carboxylmethyl cellulose (CMC), carrageenan, or xanthan gum; any anionic polycarboxylate; any salts, such as sodium fluoride anticaries agents; and any sweeteners.

The resultant mixture is agitated until a homogeneous gel phase is formed. Into the gel phase are added any pigments utilized, such as TiO₂, and additionally any acid or base required to adjust the pH of the composition. These ingredients are mixed until a homogeneous phase is obtained.

The mixture is then transferred to a high speed/vacuum mixer, wherein the inorganic thickener, such as Zeodent 165, and the surfactant ingredients are added to the mixture. Any abrasives utilized are added at this point. Any water insoluble bacterial agents, such as Triclosan, are solubilized in the flavor oils to be included in the dentifrice, and that solution is added along with the surfactants to the mixture, which is then mixed at high speed for about 5 to about 30 minutes, under a vacuum of about 20 to about 50 mm of Hg, specifically about 30 mm Hg. The resultant product is a homogeneous, semi-solid, extrudable paste or gel product.

In other embodiments, the present invention provides methods of reducing inflammation and inhibiting gingivitis, comprising applying a composition comprising a Hippophae extract to the oral surfaces.

In other embodiments, the present invention provides methods of improving oral hygiene comprising the application of a composition comprising a Hippophae extract to the oral surfaces.

As used herein, “applying” includes actions normally associated with oral care, and includes, e.g., manually rubbing or dabbing, rubbing with an implement such as a sponge or dental instrument, brushing, irrigating, rinsing, spraying, wiping, painting, flossing, gargling, placement of a film or strip on the surface, implanting and chewing.

The following Examples further describe and demonstrate embodiments within the scope of the present invention by way of example, and not limitation. The Examples are given solely for illustration purposes and are not to be construed as a limitation of the present invention, as many variations thereof are possible without departing from its spirit and scope.

### EXAMPLES

The following are examples of oral care compositions that are prepared within the parameters of the present invention. These compositions, when used on a regular basis, are effective in cleaning teeth and in providing various benefits to the user.

### Example 1

A dentifrice composition with the following components is prepared:

| Constituent | Weight Percentage Range |
|---|---|
| Precipitated Calcium Carbonate | 20–60 |
| Purified Water | q.s. |
| Sorbitol (70% solution) | 15–50 |
| Sodium Lauryl Sulfate (35% liquid) | 1–10 |
| Sodium Monofluorophosphate | 0.1–5 |
| Sodium CMC - 500T | 0.1–3 |
| N-silicate | 0.1–3 |
| Xanthan Gum | 0.05–3 |
| Sodium Saccharine | 0.01–3 |
| Sodium Tetrapyrophosphate | 0.01–3 |
| Methyl-parahydroxybenzoate | 0.01–3 |
| Colorant (Pigment and Dye) | 0.001–2 |
| Propyl-parahydroxybenzoate | 0.001–1 |
| Hippophae Extract | 0.001–20 |
| Chamomile Glycolic Extract | 0.001–5 |
| Flavoring Agent | 0.01–5 |
| Sage Extract | 0.001–5 |
| Tea Tree Oil | 0.001–1 |

### Example 2

A dentifrice composition with the following components is prepared:

| Constituent | Weight Percentage Range |
|---|---|
| Purified Water | q.s. |
| Sorbitol (70% solution) | 40–90 |
| Hydrated Silica | 4–20 |
| Silicon Dioxide | 4–20 |
| PEG 600 | 0.01–5 |
| Sodium Lauryl Sulfate (35% liquid) | 1–10 |
| Sodium Monofluorophosphate | 0.1–5 |
| Sodium CMC - 500T | 0.1–3 |
| Calcium Glycerophosphate | 0.001–1 |
| Sodium Saccharine | 0.01–3 |
| Sodium Tetrapyrophosphate | 0.01–3 |
| Colorant (Pigment and Dye) | 0.001–2 |
| Hippophae Extract | 0.001–20 |
| Chamomile Glycolic Extract | 0.001–5 |
| Flavoring Agent | 0.01–5 |
| Sage Extract | 0.001–5 |
| Tea Tree Oil | 0.001–1 |

### Example 3

A dentifrice composition with the following components is prepared:

| Constituent | Weight Percentage Range |
|---|---|
| Purified Water | q.s. |
| Sorbitol (70% solution) | 20–60 |
| Hydrated Silica | 4–20 |
| Silicon Dioxide | 2–10 |
| PEG 600 | 0.01–5 |
| Sodium Lauryl Sulfate (35% liquid) | 1–10 |
| Sodium Monofluorophosphate | 0.1–5 |
| Sodium CMC - 500T | 0.1–3 |
| Calcium Glycerophosphate | 0.001–1 |
| Xanthan Gum | 0.05–1 |
| Sodium Saccharine | 0.01–3 |
| Sodium Tetrapyrophosphate | 0.01–3 |
| Titanium dioxide | 0.05–2 |
| Methyl-parahydroxybenzoate | 0.01–3 |
| Colorant (Pigment and Dye) | 0.001–2 |
| Propyl-parahydroxybenzoate | 0.001–1 |
| Hippophae Extract | 0.001–20 |
| Chamomile Glycolic Extract | 0.001–5 |
| Flavoring Agent | 0.01–5 |
| Sage Extract | 0.001–5 |
| Tea Tree Oil | 0.001–1 |

### Example 4

A dental floss composition with the following components is prepared:

| Component | Weight Percentage Range |
|---|---|
| PTFE Yarn | 70–90 |
| Microcrystalline Wax | 9–15 |
| Hippophae Extract | 0.05–20 |

### Example 5

A mouthwash composition with the following components is prepared:

| Component | Weight Percentage Range |
|---|---|
| Demineralized Water | 60–85 |
| 99% Glycerine (USP) | 5–10 |
| 70% Sorbitol Solution | 3–10 |
| Propylene Glycol | 3–10 |
| 95% Ethyl Alcohol | 4–10 |
| Cetylpyridinium Chloride | 0.01–1 |
| Sodium Saccharin (USP) | 0.001–0.5 |
| Sodium Fluoride (USP) | 0.01–1 |
| Polysorbate 20 | 0.01–2 |
| Flavor | 0.05–3 |
| Hippophae Extract | 0.001–20 |

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims. For example, the present invention can be used in a variety of portable forms for a variety of compositions.

1. An oral care composition comprising:
(a) about 0.001 to about 20% of a Hippophae extract;
(b) an acceptable carrier; and
(c) a calcium ion donor.

2. The composition according to claim 1, wherein the extract is an extract of Hippophae rhamnoides.

3. (canceled)

4. The composition according to claim 1, wherein the Hippophae extract is present in an amount of about 0.002 to about 15%.

5. The composition according to claim 1, wherein the Hippophae extract is present in an amount of about 0.005 to about 12%.

6. The composition according to claim 1, wherein the calcium ion donor is present in amounts of about 0.001 to about 60%.

7. The composition according to claim 1, wherein the calcium ion donor is a calcium salt.

8. The composition according to claim 7, wherein the calcium ion donor is chosen from calcium glycerophosphate, calcium phosphate, calcium gluconate, calcium carbonate, calcium chloride, calcium sulfate, calcium oxide or mixtures thereof.

9. The composition according to claim 1, in the form of a dentifrice, lozenge, gum, paste, gel, dissolvable or undissolvable film.

10. The composition according to claim 9, in the form of a toothpaste.

11. The composition according to claim 9, in the form of a mouthwash or mouthrinse.

12. A dental floss comprising a composition according to claim 1.

13. The dental floss composition according to claim 12, further comprising a wax.

14. The composition according to claim 1, further comprising a fluoride ion source.

15. The dentifrice composition according to claim 9, further comprising a surfactant.

16. The composition according to claim 1, further comprising an active agent chosen from antioxidants, vitamins, plant extracts or mixtures thereof.

17. The dentifrice composition according to claim 9, further comprising about 5% to about 75% of a humectant selected from glycerin, sorbitol, propylene glycol, and mixtures thereof.

18. The composition according to claim 1, further comprising an anticalculus or antibacterial agent, or mixtures thereof.

19. The composition according to claim 1, further comprising an anionic polycarboxylate polymer.

20. A method of reducing inflammation of the oral tissues comprising applying a composition according to claim 1 to the oral surfaces.

21. A method of inhibiting gingivitis comprising applying a composition according to claim 1 to the oral surfaces.

22. A method of reducing the prevalence of plaque comprising applying a composition according to claim 1 to the oral surfaces.

23. A method of maintaining or increasing systemic health of a mammal comprising applying a composition according to claim 1 to the oral surfaces at least once a day for a duration of time.
